(19) United States
(12) Patent Application Publication
(10) Pub. No.: US 2014/0370593 A1
(43) Pub. Date: Dec. 18, 2014

(54) FLOW CHAMBER ASSEMBLY AND METHODS OF USING THE SAME

(71) Applicant: DUKE UNIVERSITY, Durham, NC (US)

(72) Inventors: Hardean E. Achneck, Durham, NC (US); George A. Truskey, Durham, NC (US); Steven J. Owen, Durham, NC (US)

(73) Assignee: DUKE UNIVERSITY, Durham, NC (US)

(21) Appl. No.: 14/363,897

(22) PCT Filed: Dec. 10, 2012
(86) PCT No.: PCT/US12/68781

§ 371 Date: Jun. 9, 2014

Related U.S. Application Data
(60) Provisional application No. 61/568,252, filed on Dec. 8, 2011.

(51) Int. Cl. C12M 3/00; C12M 1/26

(52) U.S. Cl. CPC C12M 23/44; C12M 33/12
USPC 435/325; 435/305.4

(57) ABSTRACT

A flow chamber assembly for subjecting cells or other biological reagents to laminar flow conditions and methods of using the flow chamber assembly are provided herein. The flow chamber assembly includes a bottom plate having at least one well with a bottom surface adapted to receive the cells or biological reagents, a top plate having at least one flow protrusion positioned and shaped to fit into the well of the bottom plate and a sealing element positioned between the top plate and the bottom plate when the top plate and the bottom plate are attached. The flow chamber assembly is configured to allow for laminar flow of a perfusate across the cells or biological reagents along the bottom surface of the well of the bottom plate. The cells or biological reagents can be exposed to a predetermined level of shear stress.

### CROSS-REFERENCE TO RELATED APPLICATIONS

This patent application claims the benefit of priority of U.S. Provisional Patent Application No. 61/568,252, filed Dec. 8, 2012, which is incorporated herein by reference in its entirety.

### FIELD OF THE INVENTION

The invention described herein relates to flow chamber assemblies and systems and methods of using the same. More specifically, the invention relates to a flow chamber assembly that allows cells or biologic, reagents to be subjected to laminar flow conditions and to provide a predetermined level of shear stress to the cells or biological reagents. Suitably the flow chamber assembly is suitable for use with a microscope to allow viewing of cells or biological reagents under flow conditions and allows the cells or biological reagents to be collected or harvested from the flow chamber assembly after exposure to flow conditions.

### INTRODUCTION

Many cells in the human body are constantly subjected to fluid shear stresses. Endothelial cells, for example, line the inner surface of blood vessels and are exposed to fluid flow. in order to study the differentiation and behavior of such cells under well quantifiable fluid shear stresses and different conditions, flow chambers are needed. The flow chamber design described herein emerged from a need to subject adherent cells or other biological reagents to laminar flow conditions and evaluate their response to well-quantifiable fluid shear stresses. While some flow chambers are currently available, they all have various limitations.

Initially flow chambers were designed by compressing a gasket between two parallel flat plates (GlycoTech); however, the height of the gasket changes based on the force applied to the plates. Therefore the height varies with every experiment and the shear stress is not constant or quantifiable. Similar designs (FlexFlow™) further require the use of vacuum pumps to prevent leakage of fluid during a flow experiment. These systems are further limited in that only one experimental condition can be studied per experiment. The Flexcell Streamer® was created in an attempt to overcome the latter problem. It allows several glass slides to be exposed to the same shear stress at once but lacks a viewing window for real-time visualization of cells under flow.

More recently, microfluidic flow chambers have been designed in an effort to study cells under many different conditions per single flow experiment. Ibidi's® multi-channel slide can be used for multiple flow assays in parallel and its channel contents can be visualized in real-time.

However, thee flow channels require a microscope for read-out and are not compatible with microtiter plate readers.

CellAsic engineered MiCA™ plates, which integrate their microfluidic channels in the middle of three consecutive wells of microtiter plates. In its design, the outer two wells serve as inflow and outflow reservoirs, respectively. Since gravity is responsible for the perfusion of the culture chamber in MiCA™ plates, the shear stress cannot be varied. Therefore it is not possible to model arterial, venous and capillary fluid shear stresses.

Moreover, none of these microfluidic devices are suitable to harvest a sufficiently large number of cells for DNA or RNA purification experiments after exposure to flow. They are also not convenient to use for the purification of specific proteins expressed in cells after flow, e.g. to perform a Western blot, because the microfluidic channels do not harbor sufficiently large numbers of cells. Furthermore, these devices are not suitable as replacement for cell culture plastic ware and therefore routinely require disposable plastic flasks for culturing cells prior to flow experiments. It would also be difficult to mass-produce these devices at low cost, which limits their use as a more universal research tool in life sciences. Lastly, these devices cannot function without external components, such as external pumps, to subject cells to different shear stresses, and are therefore not practical to utilize for automated processes, e.g. robotics.

### SUMMARY

A flow chamber assembly for subjecting cells or other biological reagents to laminar flow conditions and methods of using the flow chamber assembly are provided herein. The flow chamber assembly includes a bottom plate having at least one well with a bottom surface adapted to receive the cells or biological reagents, a top plate having at least one flow protrusion positioned and shaped to fit into the well of the bottom plate and a sealing element positioned between the top plate and the bottom plate when the top plate and the bottom plate are removably attached. The flow chamber assembly further includes a flow path comprising a fluid feeding channel, an inflow bay, a laminar flow section, an outflow bay and a fluid exit channel all of which are in fluid communication with each other as described and shown herein. In an alternative embodiment the top plate and the bottom plate may form a unitary construction or be attached such that the top plate and bottom plate once attached are not removably attached.

The laminar flow section of the flow chamber assembly is suitable for subjecting the cells or biological reagents to laminar flow conditions when the top plate is attached to the bottom plate and the flow chamber assembly is in operation. The flow chamber assembly is constructed such that the width, height and hydraulic diameter of the laminar flow section are known and fixed to allow for laminar flow. The flow rate, fluid density and viscosity can be altered to provide a calculated shear stress to the cells or biological reagents in the laminar flow section of the flow chamber assembly. In addition, the bottom surface of the well and the lower surface of the flow protrusion may be made of an optically clear material such that the cells or biological reagents can be monitored optically using microscopes or microtiter plate readers. The flow chamber assembly provided herein may comprise more than one set of paired wells and flow protrusions connected in series or in parallel to ensure equivalent treatment of more than one sample.

In another aspect, a top plate for a flow chamber assembly is provided. The top plate is capable of being removably attached to a bottom plate comprising wells to form a flow chamber assembly. The top plate includes at least one flow protrusion positioned and shaped to fit into the well of the bottom plate, and a flow path comprising a fluid feeding channel, an inflow funnel, an outflow funnel, and a fluid exit channel.

In another aspect, systems for subjecting cells or biological reagents to laminar flow conditions to provide a predetermined level of shear stress to the cells or biological reagents are provided. The systems include the flow chamber assembly described herein connected to an external pump capable of pumping a perfusate through the flow chamber assembly and a reservoir connected in series to the flow chamber assembly and the pump. An alternative system includes a flow chamber assembly, which includes within the flow chamber assembly a pumping mechanism capable of pumping a perfusate through the flow chamber assembly and a reservoir, Thus, the flow chamber assembly can be used without external connections to the flow path. The pumping mechanism may he driven by an external motor, may be part of or positioned within the flow chamber assembly or may be external to the flow chamber assembly.

In a further aspect, methods of using the flow chamber assemblies and the systems described herein are provided. The methods include adding perfusate including the cells or biological reagents to be analyzed to the well of the bottom plate and attaching the top plate to the bottom plate of the flow chamber assembly. Then the perfusate is pumped from a reservoir through the fluid feeding channel into the inflow hay, through the laminar flow section and back out through the outflow bay and the fluid exit channel to achieve laminar flow in the laminar flow section of the flow path. The methods allow the cells or biological reagents to be exposed to a predetermined shear stress and biological properties of the cells or biological reagents to be assayed.

### BRIEF DESCRIPTION OF THE DRAWINGS

FIG. 1 is a perspective view of the multi-well flow chamber assembly when fully assembled.

FIG. 2 is a perspective view of a multi-well flow chamber assembly incorporating the concepts described herein.

FIG. 3 is a perspective view of the underside of the top plate.

FIG. 4 is a pictorial cross-sectional cut of the top plate made at line 4-4 in FIG. 3.

FIG. 5 is a pictorial cross-sectional cut of the top plate made at line 5-5 in FIG. 3.

FIG. 6 is a detailed perspective view of the bottom plate.

FIG. 7 is a longitudinal cross-section view taken along line 7-7 in FIG. 1 showing a single well and a portion of the flow path.

FIG. 8A is a longitudinal cross-section taken along line 8-8 in FIG. 7.

FIG. 8B is a longitudinal cross-section taken along line 8-8 in FIG. 7.

FIG. 9 is a perspective view of an alternative embodiment in which the flow path is contained in the bottom plate.

FIG. 10 is a cross-section taken along line 10-10 in FIG. 9.

FIG. 11 is a schematic view of a flow path connecting three wells in parallel.

FIG. 12 is a schematic of a mixing chamber as a means to connect wells to the flow path in parallel.

FIG. 13 is a side elevation view of the top and bottom plate showing interlocking teeth as a locking mechanism.

FIG. 14 is a perspective view of the flow chamber assembly in operation and connected to an external reservoir, pump and pulse dampener.

FIG. 15 is a schematic of a flow chamber assembly incorporating an impeller and a reservoir into the flow path with two wells connected in series.

FIG. 16 is a schematic of a flow chamber assembly incorporating an impeller and a reservoir into the flow path with four wells.

FIG. 17 is a schematic of a centrifugal pump that could be designed for placement below the bottom plate with the arrows representing the positioning of an inlet port and outlet port in the bottom plate.

FIG. 18 is a flow chart detailing methods of using the flow chamber assembly.

### DETAILED DESCRIPTION

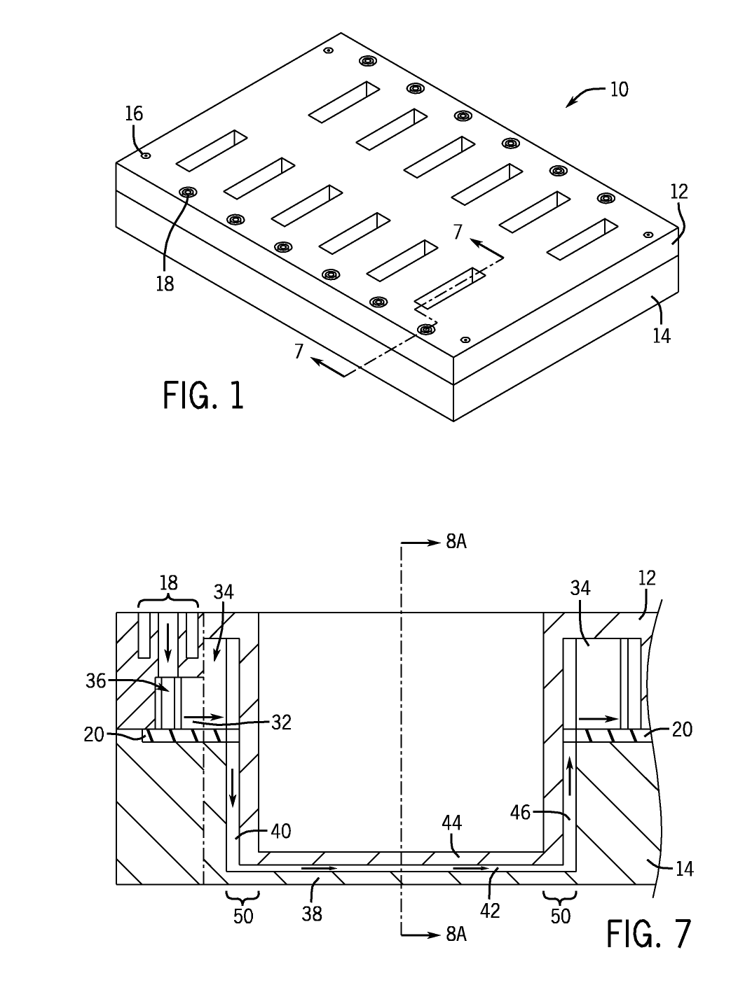

FIG. 1 illustrates a multi-well flow chamber assembly 10 that includes a top plate 12 and a bottom plate 14, which can be removably attached to each other using screws 16 or other locking mechanisms described further below. The plates contain a flow path that may be a continuous flow path completely contained within the plate or may be connected to ports 18 that connect the flow path to the outside of the flow chamber assembly. When in operation, the flow chamber assembly allows cells or other biological reagents to be exposed to laminar flow conditions in a portion of the flow path and to provide a predetermined level of shear stress to the cells or biological reagents. The flow chamber may be made of a transparent or optically clear material suitable for optically monitoring cells or other biological reagents before, during or after being exposed to flow conditions. The flow chamber assemblies described herein are compatible for use with microscopes, microtiter plate readers and robotics. The flow chamber assembly generally has a similar size and shape as traditional microtiter plates. Several advantages of one or more aspects of one or more of the flow chamber assemblies provided herein include providing a flow chamber with constant height of the flow path, that does not require vacuum pumps for sealing and does not leak, that enables real-time visualization of cells under flow and has multiple wells to enable the use and direct comparison of different cell types, different reagents or bioassays or drugs in different wells. Suitably, the flow chamber assembly enables the use of different flow conditions and shear stresses per experiment and subjects sufficiently large numbers of cells to fluid shear stress to enable a scientist to harvest cells' DNA or RNA, to conduct PCR, RT-PCR or microarray-based follow-up experiments, or to analyze cells' protein expression with a Western blot or other protein-based bioassay after flow.

The bottom plate 14 can be utilized for cell culture with a traditional microtiter top plate and thus can replace use of tissue-culture flasks to prepare cells before exposure to flow conditions. Thus the flow chamber assembly provided herein will reduce the steps in the routine work-flow. The flow chamber assembly is designed to be mass-produced at low cost via injection molding. Other advantages of one or more aspects will be apparent from a consideration of the drawings and ensuing description.

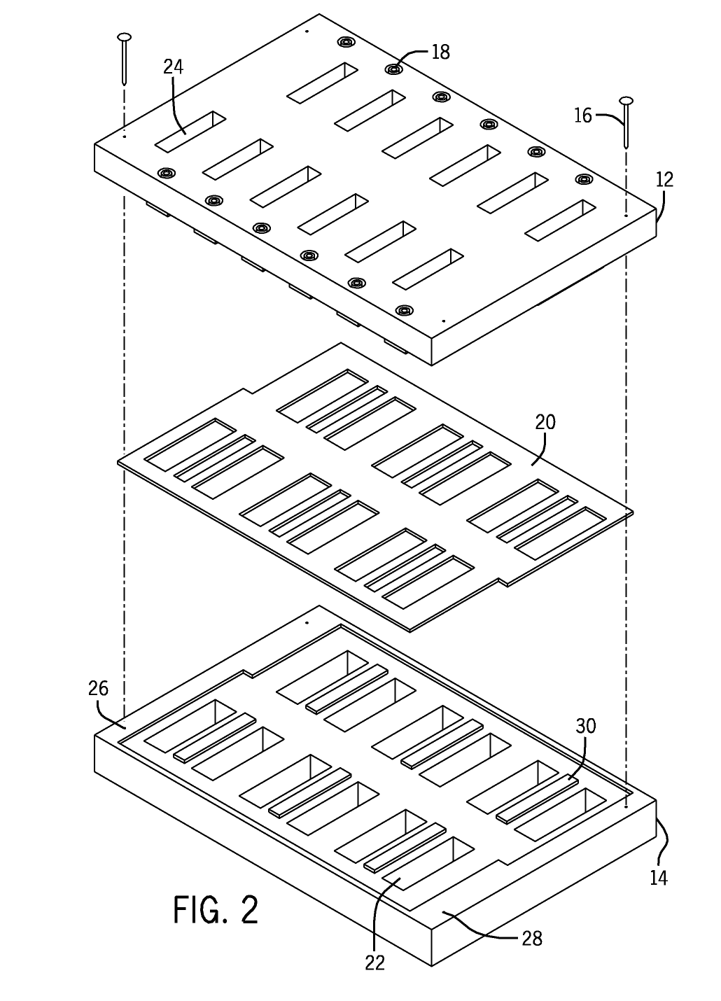

With reference to FIG. 2, the flow chamber assembly includes a top plate 12, a bottom plate 14 and a sealing element, shown as a gasket 20 in FIG. 2. The bottom plate 14 contains a plurality of wells 22 and the top plate 12 has a corresponding plurality of flow protrusions 24 positioned and shaped to fit into the wells 22 of the bottom plate to form the flow chamber assembly 10 shown in FIG. 1. In FIG. 2, the wells 22 and flow protrusions 24 are rectangular in shape, but other shapes may be utilized including circular shapes like those found in traditional microtiter plates. The flow protrusions 24 are shaped to fit into the well 22 such that when the flow chamber assembly 10 is in operation the top plate is in direct contact with the bottom plate and the protrusions extend into the wells defining portions of the flow path. The flow chamber assembly is constructed in such a way that when it is assembled a flow path is defined which allows for a perfusate to travel through the array of wells 22 in the bottom plate 14. The flow chamber assemblies are leak-proof, easy to use, and suitably disposable. They may be provided in sterile packages.

The sealing element, shown as a gasket 20 in FIG. 2, is positioned between the top plate 12 and the bottom plate 14 and allows for fluid communication between the top plate and the bottom plate in the flow path, but provides a fluid tight seal for the flow chamber assembly as a whole when it is in operation. When the top plate and the bottom plate are removably attached e.g. using the screws 16 and corresponding threaded receivers 26, the gasket 20 may be received by a rim 28 on the bottom plate. The rim 28 defines the positioning and spacing of the gasket or sealing element. The bottom plate optionally also contains bumpers 30 which pass through the gasket and make direct contact between the top plate and the bottom plate to ensure the proper height of the flow path. In an alternative embodiment, the gasket 20 may also provide the appropriate spacing between the top plate and the bottom plate to define the flow path.

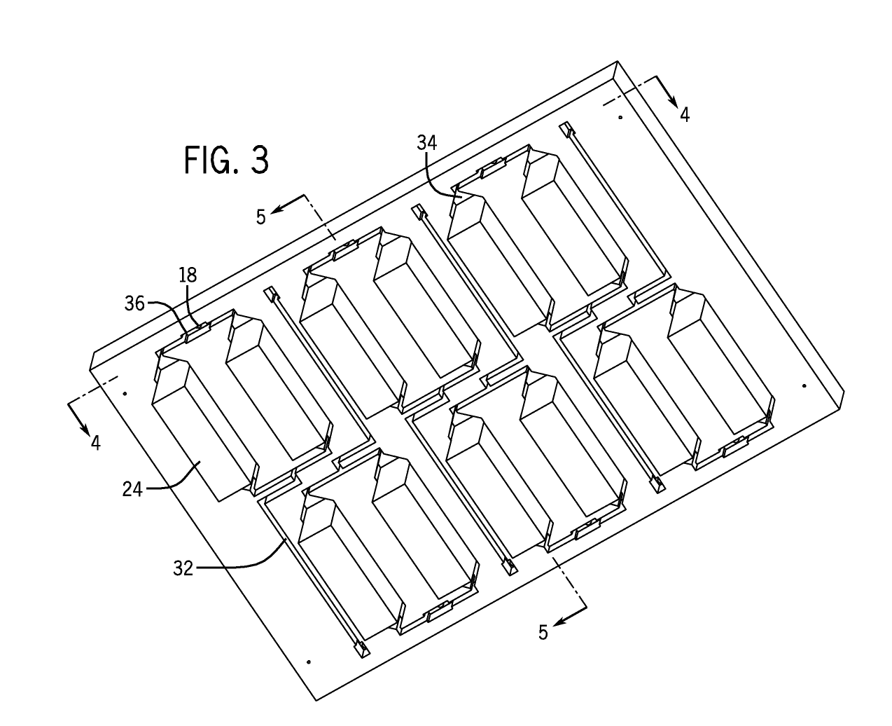

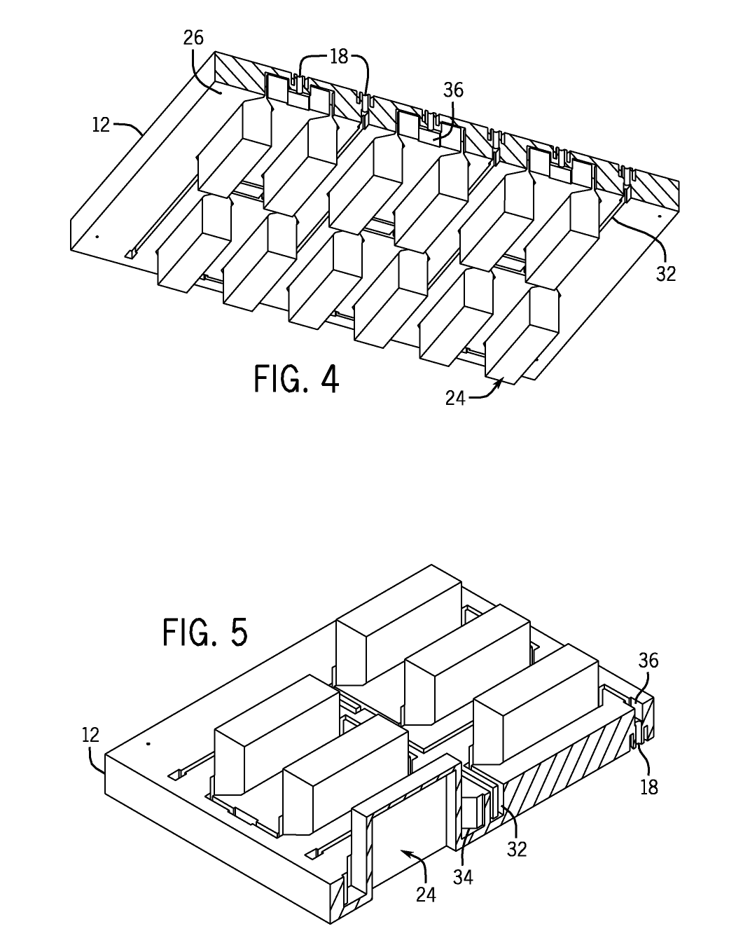

FIGS. 3-6 are directed to an embodiment of the flow chamber assembly in which the majority of the flow path is contained within the top plate 12. FIG. 3 shows the lower surface of the top plate 12 with the flow protrusions 24 connected to fluid feeding channels 32 via a funnel 34. The fluid feeding channels 32 are connected to the ports 18. FIGS. 4 and 5 provide pictorial cross-sectional cuts of the top plate 12 to more clearly show the design of the ports 18, which allow connection of the flow chamber assembly to tubing to supply the perfusate to the flow chamber assembly 10 and its connection to the fluid feeding channels 32 and the funnels 34.

In FIG. 3, two flow protrusions 24 are connected in parallel to the fluid feeding channels. In this embodiment, a perfusate will enter the flow chamber assembly 10 via a port 18, the inlet port, and be directed via a fluid feeding channel 32 towards two protrusions 24 and their corresponding wells 22 by entering via the funnel 34. The fluid feeding channel may have a Y-junction to split into separate fluid feeding channels 32 for the two flow protrusions 24. The perfusate will exit via the funnel 34 at the opposite end of the flow protrusion 24, and travel back out of the flow chamber assembly via two fluid feeding channels 32 acting as fluid exit channels that optionally converge into one fluid feeding channel. A basin 36 is optionally located where the two short feeding channels converge near the port 18 and the perfusate exits the flow chamber assembly at an outlet port 18. A basin 36 may be located at the end of or within a fluid feeding channel 32. In FIGS. 3-6, basins 36 are shown at the end of the fluid feeding channels 32 at the port 18. Notably, the fluid flow path is reversible, such that the perfusate can enter at any port 18, flow through the fluid feeding channel 32 into the funnel 34 at one end of the flow protrusion 24 and back out at the funnel 34 at the opposite end of the flow protrusion 24, through the connected fluid feeding channel 32 and out the port 18 at the end of the fluid feeding channel. In operation, the flow chamber assembly ports 18 will serve as either an inlet port for fluid entry or as an outlet port for fluid exit. The fluid feeding channels 32 will likewise serve as either a fluid feeding channel that will deliver perfusate towards the funnel 34 and protrusion 24 or will act as a fluid exit channel taking the perfusate out of the flow chamber assembly.

The port 18 (including both the inlet port and the outlet port) is designed to connect to the fluid feeding channels 32 on the outside surface of the top plate 12 (FIG. 2 and 3-5). It allows for connection of flexible tubing to allow entry of the perfusate into the flow chamber assembly.

Since the height of the flow chamber assembly may be restricted such that the assembled chamber fits under a condenser of a microscope on a microscope stage, the fluid feeding channel opposite to the port in this embodiment is <0.7 cm deep. To maintain the condition that the major resistance in the flow chamber assembly shall occur in the wells 22 in the laminar flow section, the area opposite the port may be wider to compensate for the decrease in depth, e.g. 0.2 cm wide and 0.4 cm deep. This area is denoted as basins 36 in FIGS. 3, 4, and 5. While the ports are shown as on the upper surface of the top plate, other configurations are available such as the sides of the top plate or the bottom plate as will be more fully described later.

With reference to FIG. 6, the bottom plate 14 has a plurality of wells 22 that correspond to the flow protrusions 24 in the top plate 12. The wells are large enough to hold cell culture medium sufficient to culture cells inside the wells. The bottom surface 38 of the well is suitable for culturing cells and is tissue-culture treated. Tissue culture treatment of microtiter plates is well known in the art and may be achieved with oxygen plasma discharge modification or a similar technology employed to coat tissue culture plastic ware for the purpose of improving cell adhesion and attachment. Cells can he cultured under static conditions initially, e.g. in a humidified incubator at 37° C. and 5% CO₂, 21% O₂. Each well may be filled with a desired cell type and different drugs or drug combinations. After culture under the desired conditions, the cells can be exposed to laminar flow fluid shear stress by adding the top plate onto the bottom plate. Alternatively, cells can be cultured under flow conditions and added to the wells under flow conditions. For this purpose, the top plate is added to the bottom plate at the beginning of an experiment. To keep cells sterile during culture, a lid can be manufactured that tits tightly onto the bottom plate but still leaves enough room to allow for gas exchange between the individual wells and the incubator environment.

The bottom plate 14 is of such geometry and dimensions that it can be inserted into standard microtiter plate readers. The wells 22 are arranged in such pattern that the light beams of a microtiter plate reader transverses the wells. For example, the wells may be spaced such that the well center to well center distance is a multiple of 9 mm consistent with use in a 96 well microtiter plate reader. Microtiter plate readers can detect biological, chemical and physical events of samples in the array of wells in the bottom plate of the high-throughput multi-well flow chamber assembly. The high-throughput flow chamber assembly may be used for assays that are based on but not limited to the detection of and quantification of time-resolved fluorescence energy transfer, time-resolved fluorescence, fluorescence resonance energy transfer, fluorescence intensity, fluorescence polarization, bioluminescence resonance energy transfer, luminescence detection, spectrometer absorbance, alpha screen, simultaneous dual emission, bottom reading, etc. Therefore, the bottom plate 14 of the high-throughput flow chamber assembly can be used analogous to a standard microtiter plate.

The bottom plate 14 may further comprise bumpers 30 and a rim 28, which define an elevation over the remaining surface of the bottom plate. In one embodiment, this remaining surface of the bottom plate is covered with a gasket 20 as shown in FIG. 2. In an alternative embodiment the sealing element may be overmolded onto the plastic of the bottom plate 14. This would reduce the number of parts to assemble the flow chamber assembly and help maintain sterile conditions and ease of use. The gasket may be made of silicone-rubber. Liquid silicone rubber is commonly used in overmolded plastic parts to produce seals or sealing membranes or elements. When the flow chamber assembly is in operation, the top plate 12 is firmly pressed against the bottom plate 14 and the gasket 20 is compressed such that the rim 28 and bumper 30 of the bottom plate touch the undersurface of the top plate. A person skilled in the art of engineering can determine the hardness (durometer) of the silicon-rubber gasket to ensure that during full compression of the flow chamber assembly with a predetermined force the material of the top plate is in direct contact with the elevated material of the bottom plate.

The top plate 12 and the bottom plate 14 may be made such that the orientation of the top plate relative to the bottom plate is maintained. This directionality may be achieved through various means for example, one or more of the threaded holes for receiving a screw during attachment of the top plate to the bottom plate may be offset as compared to the others so that to align the threaded hole in the bottom plate with the hole in the top plate the top and bottom plate must be oriented to each other in the same configuration. In this embodiment, if the top plate was rotated by 180 degrees with respect to the bottom plate the holes would not align and the screws could not be used to affect attachment of the top plate and the bottom plate. In an alternative embodiment, the edge or corner of one side of the top plate may be rounded or have a shape distinct from the other edges or corners and the bottom plate has a corresponding rounded edge or corner such that the two plates only fit together in one orientation. This strategy for maintaining directionality of fit between a bottom plate and a top plate is commonly found in commercially available microtiter plates. The gasket may also be fitted with a rounded corner or edge similar to that of the top and bottom plates. In another alternative, the wells 22 of the bottom plate and the flow protrusions 24 of the top plate may be configured within the plates such that the top plate flow protrusions 24 can only fit into the wells 22 of the bottom plate in one orientation. Finally, the gasket 20 may be designed with an opening and either the top plate or the bottom plate may have a bumper or pin that extends through the opening in the gasket and engages with a receiver on the opposite plate to allow for directionality in the orientation of the top plate and the bottom plate.

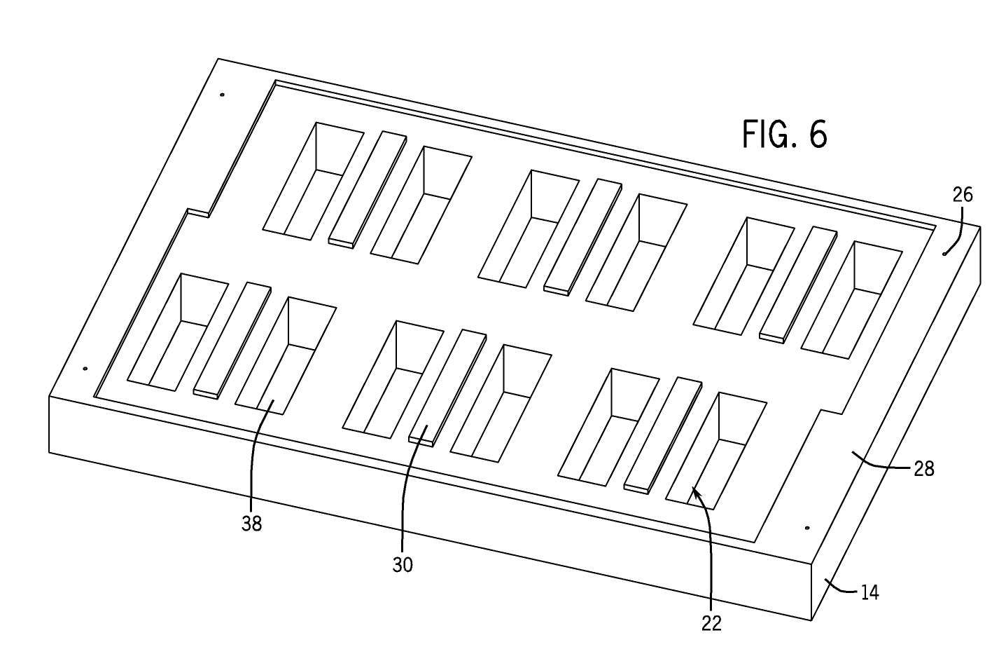

With reference now to FIG. 7, which shows a cross-sectional view of the flow path through a single well 22 of the flow chamber assembly during operation, the flow path includes an inlet port 18, a fluid feeding channel 32, an inflow bay 40, a laminar flow section 42, an outflow bay 46, a fluid exit channel and an outlet port. In FIG. 7, the arrows show the direction of flow of the perfusate in one exemplary embodiment. As noted above, the perfusate can flow in either direction through the flow chamber. The inlet port 18 is connected to the fluid feeding channel 32 which is connected to the inflow bay 40. The inflow bay is formed between a first side of the well 22, a first side of the flow protrusion 24 and the funnel 34 at the first side of the flow protrusion when the top plate and the bottom plate are attached. The inflow bay 40 is connected to the laminar flow section 42 formed by the lower surface of the flow protrusion 44 and the bottom surface of the well 38 when the top plate and the bottom plate are attached, The laminar flow section 42 is connected to the outflow bay 46 formed between a second side wall of the well, a second side of the flow protrusion and the funnel at the second side of the flow protrusion when the top plate and the bottom plate are attached. The outflow bay 46 is connected to the fluid exit channel which is the fluid feeding channel 32 carrying perfusate away from the laminar flow section 42. The fluid exit channel is connected to a basin 36 or to the outlet port 18. When a perfusate is added to the flow chamber assembly via the fluid feeding channel the perfusate will flow to the inflow bay, through the laminar flow section and continue flowing out the outflow bay, and through the fluid exit channel.

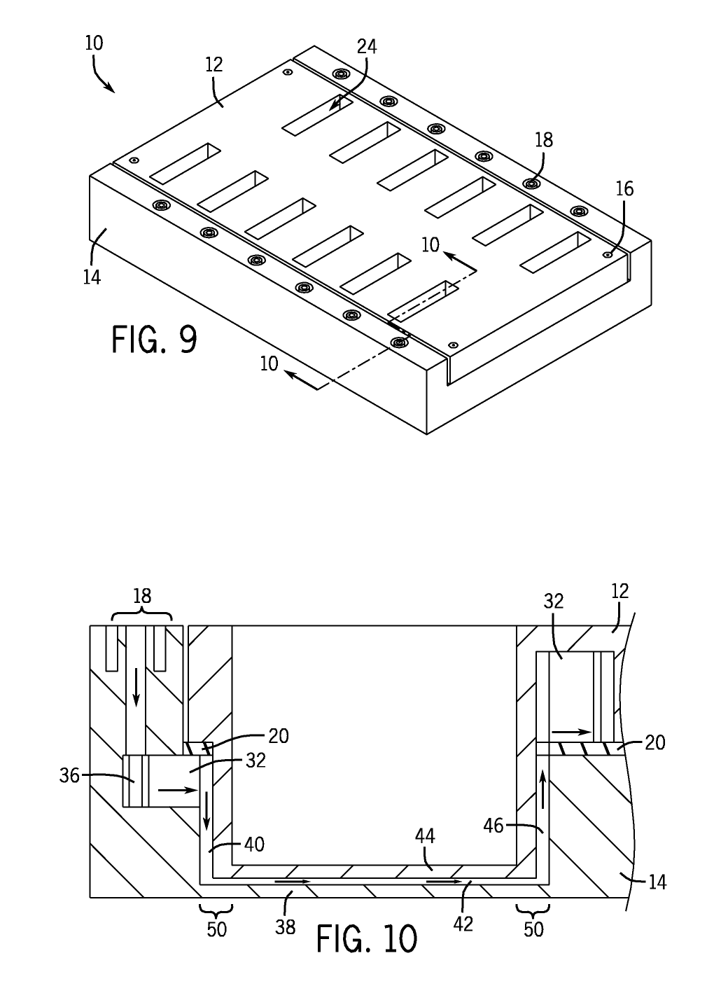

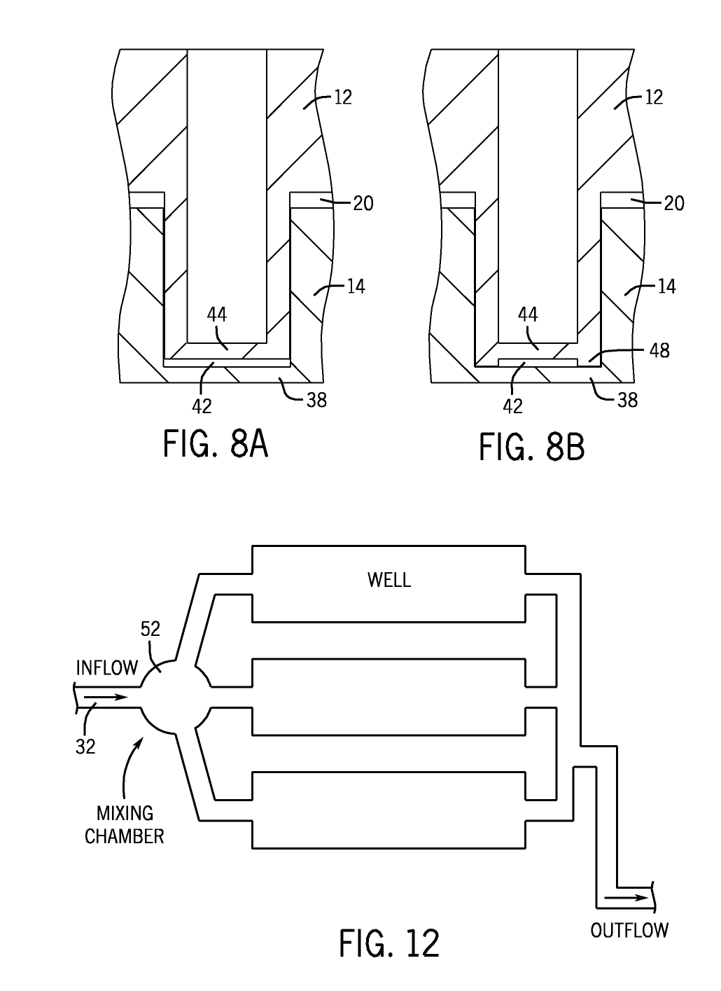

FIG. 8 shows the cross-section of two embodiments of the laminar flow section 42 of the flow chamber assembly. FIG. 8A shows the flow protrusion 24 resting in the well 22 with the flow chamber assembly in operation with the lower surface of the flow protrusion 44 and the bottom surface of the well 38 defining a space that is the laminar flow section 42. In this embodiment, the proper height of the laminar flow section 42 is maintained by the attachment of the top plate to the bottom plate, the gasket or other sealing element and the rim or bumpers described above. FIG. 8B shows an alternative embodiment in which the flow protrusion 24 comprises two or more feet 48 which extend beyond the lower surface of the flow protrusion 44 and contact the bottom surface of the well 38 when the flow chamber assembly is in operation. The feet 48 then determine the height of the laminar section of the flow path. The feet may be designed such that there are two feet that. traverse the entire length of the laminar section of the flow path. Alternatively, the feet may be present at each corner of the laminar section of the flow path such that four feet are used or only two feet at opposite corners of the laminar flow section. Those of skill in the art can envision using any number of feet to achieve the required height of the laminar section of the flow path. in an embodiment where feet on the flow protrusion are used to delineate the height of the flow path, the bumpers and rims described above may not be needed and the primary purpose of the gasket or sealing element may be limited to providing the flow chamber assembly with a seal.

FIGS. 9 and 10 show an alternative embodiment in which the flow path of the flow chamber assembly is largely contained in the bottom plate rather than the top plate. In this embodiment the ports 18 are contained in the bottom plate 14 and the top plate 12 may rest inside of the bottom plate as depicted in FIG. 9. As shown in FIG. 10, the flow path enters the flow chamber assembly at the port 18, then travels through the fluid feeding channel 32 and the inflow bay 40 into the laminar flow section 42 of the flow chamber assembly. The perfusate then exits the well 22 via the outflow bay 46 and continues through a fluid exit channel.

Initially, it is important to understand that in order to achieve laminar flow in the laminar flow section, the flow chamber assembly must be designed such that several important conditions are met. First, the flow must be laminar, which can be verified by calculating its Reynolds number (Re), which is the ratio of inertial forces to viscous forces. If viscous forces predominate, Re is small and the flow is laminar or ‘fully developed’—usually for Re <2300. When inertial forces predominate, the flow becomes more and more random until it is turbulent, as is the case for Re>4000. We can calculate Re according to equation 1:

$$Re = \frac{ρ \cdot Q \cdot {D}_{h}}{μ \cdot w \cdot h} \qquad ( 1 )$$

Where ρ is the fluid density, Q is the flow rate, μ is the viscosity, w and h are the width and height of the chamber, respectively, and Dₕ is the hydraulic diameter, defined according to equation 2:

$${D}_{h} = \frac{4 \cdot Cross \text{-} Sectional Area}{Wetted Perimeter} = \frac{4 w \cdot h}{2 \cdot ( w + h )} \qquad ( 2 )$$

Second, for the velocity field and shear stress to be independent of the distance along the laminar flow section (i.e. fully developed), the distance from the inlet bay to the laminar flow section must be longer than the entrance length 50, Lₑ, shown in FIG. 7. This can be satisfied by calculating the entrance length, according to equation 3:

Lₑ=0.04 hRₑ

Thus, a section of the bottom surface of the well is not part of the laminar flow section of the flow chamber assembly, but is the entrance length and the length can be calculated using the above formula.

The portion of the bottom surface of the well in which the flow is not laminar can be excluded during analysis of cells or biological reagents using microscopes or microtiter plate readers or can be excluded when adding cells or other biological reagents to the wells. Several other alternative strategies may be employed to avoid a possible confounding effect of non-laminar or not fully developed flow on adherent cells at the bottom of each well. The flow chamber can he constructed in such a fashion that the total length of the well, L, is much larger than the inflow length, Le (L>>Le). In the flow chamber of FIG. 7, this criterion is met with L=3 cm and Le=0.025 cm. The entrance length may not be tissue culture coated such that the cells do not adhere to this section of the bottom surface of the well. Another alternative is to ensure that adherent cells are only grown in the area where the flow path resulting from the addition of the top plate to the bottom plate is laminar and fully developed. This may be accomplished by inserting spacers into the individual wells. The spacers extend from the vertical walls of the well at the inflow and outflow bays towards the middle of the well by the length Lₑ and are in immediate contact with the side walls (see FIG. 7, Le 50). Each well may contain two spacers of equal geometry and on opposite sides of the well, one at the inflow bay and one at the outflow bay. These spacers may be constructed from the same material as the top and bottom plate of the chamber, or from other flexible material, such as rubber, silicone, etc. All spacers in all wells may be connected such that they can be easily removed en bloc as one structure. The spacers are all connected with each other such that they form a ‘grid,’ which can be removed as ‘one piece.’ The bottom plate and including the spacers is used to seed adherent cells. Prior to initiation of flow, the grid is removed as ‘one piece,’ the top plate is inserted and the flow is initiated.

Third, in order to ensure that the velocity and shear stress in the lateral direction do not vary significantly from the value for one-dimensional channel flow (ΔPh/2L), the ratio h/w must be much less than 1. For the average wall shear stress under two-dimensional flow conditions to be 95% of the wall shear stress under one-dimensional flow, h/w must be equal to 0.10, and for the wall shear stress under two-dimensional flow conditions to be >95% of the wall shear stress under one-dimensional flow, h/w must be equal to <0.1.

As an example these conditions are met for the following dimensions. Each individual well is 0.8 cm wide (w=0.8 cm), 1 cm deep and 2.7 cm long. The protrusions are 0.79 cm wide, 2.5 cm long and 1.05 cm tall, When the flow chamber is closed the top plate rests against the rim and bumpers of the bottom plate and a flow path of height h=0.05 cm is established. In this embodiment the bottom plate of the flow chamber is 8.5 cm wide and 12.75 cm long. It has 12 wells arranged in pairs of two. Each pair of wells is spaced apart by 1 cm. Each of these spaces is endowed with a 0.4 cm wide, 2.7 cm long and 0.1 cm tall bumper.

The flow Q through a single well, that is necessary to exert a shear stress of 15 dynes/cm² acting tangentially on cells adherent to the bottom of the well, can be calculated according to equation (4)

$$Q = \frac{τ \cdot w \cdot {h}^{2}}{6 \cdot μ} , \qquad ( 4 )$$

where Q is the desired flow rate, τ is the target shear stress acting tangentially on the cells (15 dynes/cm²), w is the width of the well (0.8 cm), h is the height of the flow path (0.05 cm), and μ is the viscosity of the perfusate (flow medium). Assuming a typical value of viscosity (μ) for medium as 0.9 cP (0.009 g·cm−1·s−1), Q in our example is =0.56 ml/s or 34 ml/min. Since the hydraulic diameter is defined according to equation (2), Dh is =0.094 cm. Therefore the Reynolds number, Re, according to equation (1) is ˜145.27, indicating fully-developed or laminar flow after the inlet length, Le. In our rectangular well Le can be calculated according to equation (3) and is =0.29 cm in this example, Since L>>Le a possible confounding effect of non-laminar or not fully developed flow on adherent cells at the bottom of each well can be avoided.

It should be understood from the above description that the specific dimensions can be varied, as long as the specified conditions described above defining laminar flow inside the wells arc met. In one embodiment, for example, the height h of the flow path may be reduced to 0.04 cm, 0.03 cm, 0.02 cm, etc. Further the viscosity μ of the medium may be altered by adding dextran to cell medium in order to decrease the flow Q necessary to achieve a desired shear stress.

A flow chamber assembly may be constructed with different numbers of wells and flow protrusions. For example, the wells may be designed with 2, 3, 4, 6, 8, 12, 16, 20, 24, 30, 36, 48 or more wells. The flow chamber assembly shown in FIG. 2 has 12 wells and 12 flow protrusions. In FIG. 2, the wells are connected in parallel to a flow path such that each set of two wells is connected to a single flow path, but other arrangements are possible and may be beneficial depending on the intended use of the flow chamber assembly.

The wells can be perfused separately, such that each well is connected to its own flow path. Alternatively, wells can be arranged in parallel (as shown in FIG. 2). If two wells are arranged in parallel, the fluid feeding channel bifurcates before leading to each well. The bifurcation can be in the form of a Y-junction in the fluid feeding channel. One advantage of this arrangement is that two samples of cells exposed to identical flow conditions (medium, drugs, shear stress) can be analyzed differently, e.g. one well may be used for immunohistochemistry and another well for mRNA isolation for gene expression studies.

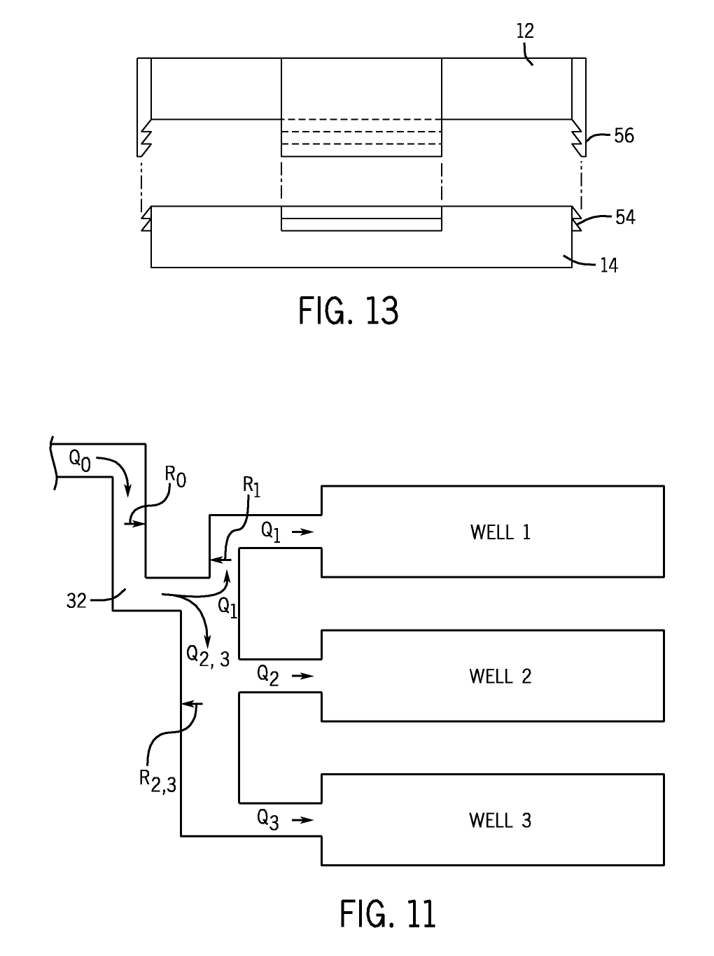

Other arrangements are possible. For example, three wells may be arranged in parallel as schematically depicted in FIG. 11. In one application of this embodiment, one well is filled with cancer cells and the other two wells are filled with two different types of ‘normal’ cells. A new cancer drug may be tested by perfusing three wells in parallel in one circuit. The desired drug should be toxic for the cancer cells, but have no toxic effects on the other two ‘normal’ cell types that are perfused in parallel under identical conditions.

In another embodiment, wells may be arranged in series, such that a compound has to transverse the first well before it can transverse the second well, etc. In this embodiment, one drug may be studied as it interacts with cells in the first well, e,g. is metabolized by hepatocytes seeded into the first well, then reaches cancer cells, which the drug is supposed to affect, and lastly reaches kidney cells to study potential nephrotoxic effects.

In designing flow chamber assemblies with various configurations of flow paths, the geometry of the fluid feeding channels must be considered. The fluid feeding channels may be round or rectangular and of such geometry that the magnitude of flow of perfusate into each well is equal. The feeding channels should be large enough so that the wells in the closed chamber represent the major resistance to the flow. In one embodiment the fluid feeding channels in the top plate are 0.1 cm wide and 0.7 cm deep. Such dimensions represent approximately 4% of the resistance to the flow through the individual wells. Therefore the above condition is satisfied.

In an alternative embodiment such as that shown in FIG. 3, two wells arranged in parallel with one flow path dividing into two wells, one flow path may also lead to only one well or a plurality of wells. When one fluid feeding channel feeds more than one well in parallel the shared portion of the fluid feeding channel must have a larger volume than the portion of the fluid feeding channel directed to only one well. FIG. 11 illustrates the arrangement of three wells in parallel. if more than two wells are arranged in parallel in one flow path, the geometry of the fluid feeding channels 32 can be varied such that the flows directed into each individual well are equal to each other. The fluid feeding channels may be round or rectangular. In. FIG. 11 the flow into each of the 3 rectangular wells, denoted Q1, Q2 and Q3, should be equal to each other and the sum of all flows equal to the total flow through the inflow channel, Q0, as described in equation (5):

Q₀=Q₁+Q₂+Q₃ (5)

Assuming a round fluid feeding channel, the radii of the bifurcation of the fluid feeding channel can be adjusted such that 1/3rd of the flow is directed to one well and 2/3rds of the flow is directed to the other two wells.

Let the radius of the inlet fluid feeding channel be R0, the radius of the channel directing fluid to one well be R1, and the radius directing flow to two other wells be R2,3. Since the flow through the inlet fluid feeding channel, Q0, equals the area of the inlet fluid feeding channel it traverses with velocity v0, we can write:

Q₀=πR₀² [symbol] v₀ [symbol] (a)

Further, we can express the flow in the different channels as:

Q₀=Q₁+Q_{2,3} (b)

And

Q₁=⅓Q₀ (c)

And

Q_{2,3}=⅔Q₀ (d)

We assume the velocities to be equal in the two branch points, such that:

[symbol]

v₁ [symbol] =⅓ [symbol] v₀ [symbol] (e)

and

[symbol]

v₁ [symbol] = [symbol] v_{2,3} [symbol] =⅓ [symbol] v₀ [symbol] (f)

Since Q₁=⅓Q₀, it follows that πR₁² [symbol] v₁ [symbol] =⅓πR₀² [symbol] v₀ [symbol] , and therefore, that the radius in the inflow fluid feeding channel is equal to its first branch point (FIG. 11):

R₁=R₀ (g)

Since Q_{2,3}=⅔Q₀, it follows that πR_{2,3}² [symbol] v_{2,3} [symbol] =π⅔R₀² [symbol] v₀ [symbol] and R_{2,3}²⅓ [symbol] v₀ [symbol] 2/3R₀² [symbol] v₀ [symbol] .

Therefore, the radius in the 2nd branch point is 1,4-times the radius of the inlet fluid feeding channel:

R_{2,3}=\sqrt{2}R₀ (h)

Note that the inflow bay into the wells may be constructed such that the flow fans out from the fluid feeding channel 32 into the well in the funnel section 34 of the inlet hay in order to minimize any ‘jetting’ of the flow at that point (see 34 in FIGS. 3 and 5).

Similar computational analyses can be performed for fluid feeding channels of square geometry where w0 is the inlet channel width and h0 the inlet channel height:

Q₀=w₀h₀ [symbol] v₀ [symbol] (i)

Assuming the height is constant throughout such that

h₀=h₁=h_{2,3} (j)

and the velocities are equal in the inlet fluid feeding channel and two branch points,

[symbol]

v₁ [symbol] = [symbol] v_{2,3} [symbol] = [symbol] v₀ [symbol] (k)

then

w₁=⅓w₀ (l)

And

w₂=⅔w₀ (m)

Note that with similar computational analyses, the geometry in channels with multiple branch points can be described such that each well in a multi-well flow chamber assembly receives the same magnitude of flow.

In an alternative embodiment, shown in FIG. 12, a mixing chamber is included at the intersection of the fluid feeding channel 32 and the branch points leading to wells. A mixing chamber 56 allows the perfusate to mix and can also function as a bubble trap or pulse dampener. The mixing chamber 56 can be a cylinder, dome, sphere, prism, pyramid, cone, cube, cuboid or combination thereof.

If the mixing chamber 56 is designed to function as a pulse dampener, its size can be constructed such that it stores a small amount of perfusate, dV, and has a total volume, VT. Its purpose is to dampen the puke generated by a pump that drives the perfusate through the flow chamber assembly. As an example, we can designate the minimum pressure in our circuit as PA and the maximum pressure at PB. The relationship between the pressures and volumes in the pulse dampener can be expressed with Boyle-Mariotte's law as:

P_{A}V_{A}=P_{B}V_{B}=P_{T}V_{T} (n)

Further assuming that

V_{A}=0.9 V_{T}, (o)

it follows that

P_{T}=0.9 P_{A} (p)

And assuming that

V_{B}=V_{A}−dV (q)

we can define the relationship between the total volume of the mixing chamber, the maximal and minimal pressures and the amount of perfusate stored in the chamber as:

$${V}_{T} = \frac{{P}_{B} dV}{0.9 ( {P}_{B} - {P}_{A} )} \qquad ( r )$$

Alternatively, to ‘build-in’ pulse dampeners, the flow chamber assembly may be connected via tubing with separate external pulse dampeners, which are connected in series to the inflow tubing running into the flow chamber assembly as described more fully below.

In an alternative embodiment, a pulse resembling the heartbeat may be desirable. This can be achieved by interposing a pulse wave generator into a laminar flow circuit or by directly generating a pulse wave of defined frequency and amplitude with a microprocessor controlled pump.

In order to ensure that the flow chamber assembly does not leak during flow, a variety of leak-proof closure mechanisms may be utilized. A snap seal design can be constructed by providing grooves 54 on either the top or bottom plate of the flow chamber assembly with interlocking teeth 56 that fit into corresponding grooves 54 and are located on the opposite plate of the flow chamber assembly. For example, a groove 56 in the top plate may fit into a corresponding tooth 54 in the bottom plate (FIG. 13). The dimensions of the teeth and grooves are such that the flow chamber assembly top plate and bottom plate ‘snap’ together and can therefore be ‘closed’ and ‘opened’ without any tools or moving parts. Such locking elements may be placed circumferentially around the entire flow chamber assembly as well as in the groups of wells that comprise one flow circuit. A snap seal may also be designed so that that it firmly locks into place when the top plate is pushed onto the bottom plate and that it requires breaking the lock or seal to open the chamber. Thus, the top plate and bottom plate need not be removably attached. In an alternative embodiment the top plate and the bottom plate may be of unitary construction such that any cells or other biological materials must be added to the wells via the flow path.

An alternative method of sealing the flow chamber assembly is to utilize a vacuum seal. For this purpose, a vacuum line, which is commonly available in laboratories, may be connected to either the top Or bottom plate of the flow chamber assembly. This will provide a negative pressure, which will keep the components of the flow chamber assembly tightly held together.

A screw closure device as described in relation to FIG. 2 above may also be used to tightly attach the top plate to its bottom plate. In one embodiment, self-tapping metal screws may be utilized. Screws may be constructed of the same material as the flow chamber assembly and a coin screw may be used. Coin screws are easily tightened by fitting a coin into the slot in the screws' heads and tightening. Alternatively, a pin and clamp mechanism may be used with the pin passing through the plates and the clamp locking the two plates together. Other closure mechanisms include, but are not limited to O-rings, gaskets, rubber gaskets, plugs, crown cap closure devices, labyrinth seals, interlocking seals or any other type of seals. The seal may also be reinforced with a friction fit design or interference fit design and/or standard tape that can we wrapped around top and bottom plates before initiating flow.

As noted above, the top plate 12 and the bottom plate 14 are suitably made out of transparent material. Suitably, the top plate and the bottom plate are constructed of a plastic material, In one embodiment this material is grade 1A optically-clear polystyrene. Other suitable materials may be used: The protrusions are carved out as shown in at least FIGS. 1 and 2 in order to minimize the amount of polystyrene or other material a light beam of a light microscope or plate reader has to transverse to visualize cells inside a well in the bottom plate. The top and bottom plate of the flow chamber assembly are suitably manufactured by injection molding, or by micro molding to achieve the desired tolerances of <+/−0.005 cm for the design of the components that define the flow path. Specifically the height of the laminar flow section of the flow path within the wells should be constructed as close as possible to the theoretical value since the shear stress for a given flow is inversely proportional to the square of the flow path height h (see equation (4)).

It may be necessary to use multiple components, which are molded separately, e.g. the top plate differently from the bottom plate. The bottom of the flow chamber or individual wells might be assembled separately by inserting glass cover slips as viewing windows compatible with inverted microscopes. Other manufacturing processes that may be applied include hot embossing, soft lithography, casting, ultraviolet embossing, vacuum forming, laser manufacturing and micromachining techniques.

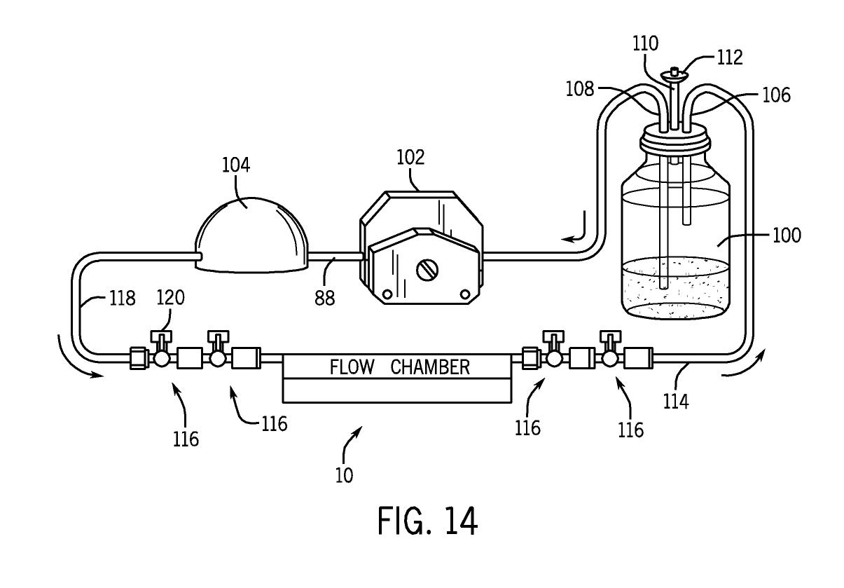

The flow chamber can be connected to a flow circuit consisting of a reservoir 100, pump 102, and flow chamber assembly 10 as depicted in FIG. 14, A pulse dampener 104 may be used in series if desired. The external reservoir 100 contains the perfusate and may consist of a hollow structure that allows for an inflow path of perfusate 106 and an outflow path 108. The inflow path 106 is designated for the inflow of perfusate into the reservoir 100 and empties into the reservoir at a higher point than the outflow path 108, such that any air is prevented from leaving the reservoir 100. The reservoir may also contain an inlet allowing for air exchange 110 and this inlet may be fitted with a filter 112 to maintain sterility of the perfusate.

For the flow chamber assembly described in FIG. 1, reservoirs 100 may be arranged in a multi-well reservoir chamber, e.g. 6-well plates with each well functioning as the reservoir for those 2 wells in the high-throughput flow chamber assembly, which have a distinct flow loop in common as shown in FIG. 3. In an exemplary embodiment of this multi-well reservoir chamber, the perfusate flows from a specific well that acts as a reservoir from the outflow path through connecting tubing into the high-throughput flow chamber assembly and feeds 2 parallel wells before exiting the high throughput flow chamber assembly in another tubing section and is then returned to the inflow side of the same well in the multi-well reservoir chamber. The six wells in the multi-well reservoir chamber correspond to 6 flow loops in parallel that simultaneously feed 6×2 wells in the high-throughput flow chamber assembly. A 6-channel pump head may be used to enable the simultaneous perfusion of the 6×2 wells. The lid of the 6 reservoir-containing plate allows for gas (and pressure) equalization with the environment. In one embodiment, the reservoir-containing plate is constructed in similar dimensions and height as the assembled flow chamber and can be conveniently placed next to the flow chamber assembly inside a standard humidified incubator. Further, the reservoir-containing plate may be designed such that the outflow paths of the wells in the flow chamber assembly directly connect with the inflow paths into the reservoir, This will minimize the circuit volume by reducing the distance the perfusate has to travel from the flow chamber assembly to the reservoirs. Each reservoir may be filled with a total volume of 1 ml or more of perfusate, e.g. medium.

The various parts of the flow circuit may be connected with microbore tubing. One piece of tubing 114 may be used to connect the flow chamber assembly 10 to the inlet 106 of the reservoir 100. A second piece of tubing 118 may be used to connect the outflow 108 of the reservoir 100 to the flow chamber assembly 10. The tubing may contain one or more stopcocks 116 or other means to control the flow of the perfusate or to gain access to the perfusate to allow sampling of the perfusate during a flow experiment. A peristaltic pump 102 can force the perfusate through the flow circuit. Other pumps known in the art may also be used in the flow circuit shown in FIG. 14. A multichannel pump head may be utilized to accommodate 6 tubes for 6 different flow circuits such as the flow paths shown in FIG. 3, which are connected to the reservoirs and 2×6 wells in the flow chamber assembly described above. A pulse dampener 104 can be constructed either as an integrated component of the flow chamber as discussed above or as an outside component in series to the flow paths to produce laminar flow. The principles underlying the calculations that determine the total volume of such a pulse dampener are provided above. Alternatively, a multi-channel syringe pump may be used to pump the perfusate through the circuit.

In one embodiment of the flow chamber assembly shown in FIG. 15, the top plate is endowed with depressions in the fluid flow path, suitably within the fluid feeding channels, that contain impellers 58. In another embodiment, the impeller is situated in the bottom plate. Theses impellers 58 work as miniature centrifugal pumps driving the perfusate in a flow loop through one or more wells (FIG. 15). The impellers 58 may be magnetic and rotated through a rotating magnetic field. Such rotating magnetic field can be produced through rotating magnets located external to the flow chamber, e.g. in depressions underneath the bottom plate directly underneath the magnetic impellers. Other pumps or pumping mechanisms may be integrated into the flow chamber to move the perfusate through the wells without the necessity for external pumps. These include, but are not limited to, piston pumps, plunger pumps, vane pumps, gear pumps, screw pumps, lobe pumps, diaphragm pumps and axial flow pumps.

The basins 36 described above, may be modified to act as internal reservoirs for the perfusate or medium may be located between wells along the flow path. Such basins 36 may be endowed with a through-hole or valve 60 to enable filling the assembled flow chamber with perfusate. In one application a robot may pipet a solution or drug into the assembled flow chamber assembly through the valve 60. In another application of this embodiment, the through-hole or valve 60 can be utilized to obtain perfusate samples at predetermined time points, e.g. via a robotically driven arm with a needle, where said needle is inserted into the through-hole or valve 60 to aspirate a small amount of perfusate for analysis of metabolites. The through-hole or valve 60 can also enable gas exchange with the outside environment if the chamber is placed inside a CO₂ incubator.

In one embodiment, a software program is utilized to control the speed of the magnetic centrifugal pumps. By modulating the rotation speed and the ‘on,’ ‘off’ frequency, different flow conditions can be recreated. These include, but are not limited to, physiological or pathological heart rhythms.

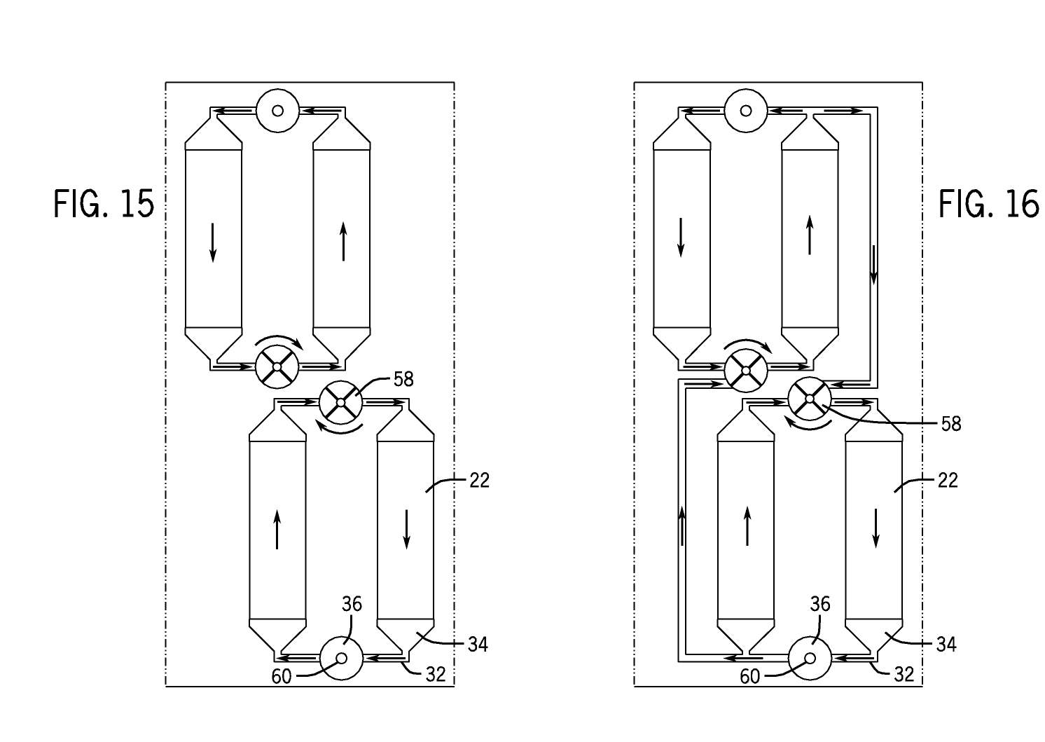

In one embodiment, the fluid feeding channels 32 of separate flow circuits are connected to create more complex circuits. One example is illustrated in FIG. 16. Complex circuits will generate different flow conditions in the individual wells and can recreate differential perfusions of different organs. The different wells of one circuit may be filled with different cell types, thus mimicking a predetermined set of organ systems. For example one flow circuit including four wells and two pumps may represent the pulmonary and systemic circuit with right and left ventricle. Through utilization of this approach, one research animal, e.g. mouse, may be replaced with one complex circuit (FIG. 16).

In another embodiment, the rotation of a motor contained within the flow chamber assembly is transmitted to the impeller 58 via direct mechanical coupling. A small output shaft may penetrate the bottom plate housing of the motor at a predetermined area and engage the impeller. A small rubber washer can provide a seal around the output shaft in order to prevent perfusate from leaking into the motor containing compartment. The impeller can be designed with a tapered opening in its central area such that an output shaft with a predetermined geometry slides into an orientation until it engages the impeller without slipping. Such mechanism will ensure that multiple impellers can be engaged to multiple output shafts simultaneously when pressing the impeller-containing plate onto a motor containing board.

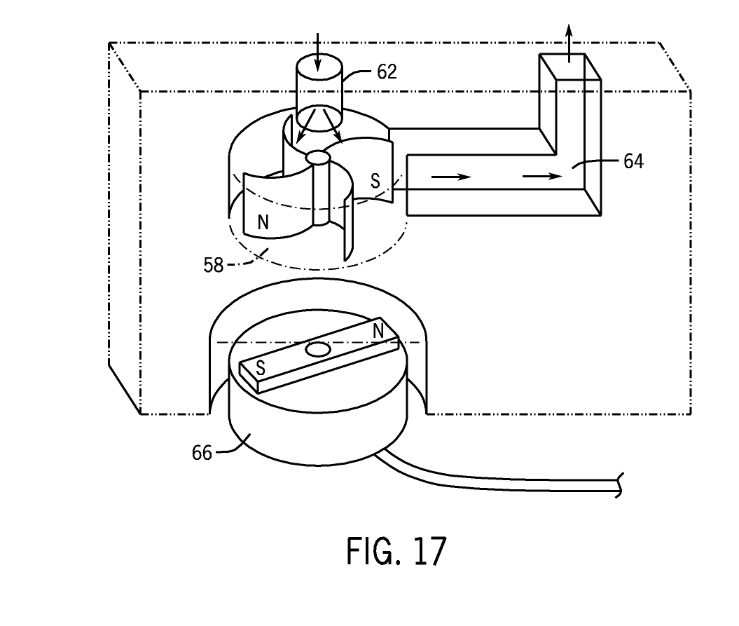

In an alternative, but related embodiment shown in FIG. 17, an inlet 62 to the impeller is located on the bottom of the bottom plate and connects the flow path to an impeller 58 located in a block below the bottom plate, the impeller 58 moves the perfusate into a discharge channel 64 which is in fluid communication with the flow path of the flow chamber assembly to allow the perfusate to reenter the flow chamber assembly. In one embodiment, the ports 18 are created through openings in the gasket material between top and bottom plate of the flow chamber assembly. The impeller 58 may be magnetically driven such that a motor 66 in the block drives the impeller and thus controls the flow rate of the perfusate into the flow chamber assembly.

The high throughput flow chamber assembly can be utilized for DNA or RNA or protein quantification of cells after exposure to shear stress. It also can be used for, but is not limited to, high-throughput drug development or screening studies, toxicological assays, chemical screening studies, screening compounds for impurities, dose-response studies, drug dissolution profile generation, drug target development, drug discovery research, evaluation of pathological states, antimicrobial compound screening, microbial adhesion studies, the evaluation of biofilm production and/or bacterial growth under different drug conditions or combinations of drugs or compounds or types of compounds under the influence of fluid shear stress, viral studies, viral infection studies, cell differentiation studies, cell morphology analyses, shear stress-depending cell differentiation studies, matrix invasion, transmigration studies, cell growth and proliferation studies, cell migration assays, basement membrane formation and degradation research, cell-cell interaction assays, cellular adhesion strength quantification, cell spreading studies, dynamic cell adhesion studies, cell rolling studies, cell-ligand interaction studies, protein-cell interaction assays, protein-protein interaction assays, including antibody binding assays, signal transduction studies, cell-signal pathway analysis, ion-flux studies, ELISAs, apoptosis assays, angiogenesis research, vasculogenesis research, cancer research, wound healing assays, thrombosis research (including platelet adhesion studies and platelet aggregation studies), further nucleofection studies, transfection studies, molecular profiling, immunoassays, lipophilicity assays, enzyme kinetic studies, kinetic turbidimetric analyses, NADH and NADPH quantification, membrane fluidity studies, colorimetric assays, solubility assays, as well as other specific applications in the fields of cardiovascular research, immunology research, and parasitology. Performance of these assays will be completed using cells or other biological reagents such as proteins, antibodies, metabolites, platelets, cellular membranes and the like.

Note that while we have described a specific arrangement of the wells in the above examples, the wells may be arranged in different patterns. Similarly, the wells may have different dimensions and geometry and may be grouped in different combinations. Therefore, the flow chamber may be designed with either one inflow and outflow channel only, or with a multitude of separate inflow and outflow channels that may correspond to a multitude of different flow circuits.

While the invention has been described in connection with what is presently considered to be the most practical and preferred embodiment, it is to be understood that the invention is not to be limited to the disclosed embodiment, but on the contrary, is intended to cover various modifications and equivalent arrangements. The following examples are meant only to be illustrative and are not meant as limitations on the scope of the invention or of the appended claims. All references cited herein are hereby incorporated by reference in their entireties.

### EXAMPLES

### Operation of Flow Chamber Assembly for Cell Culture

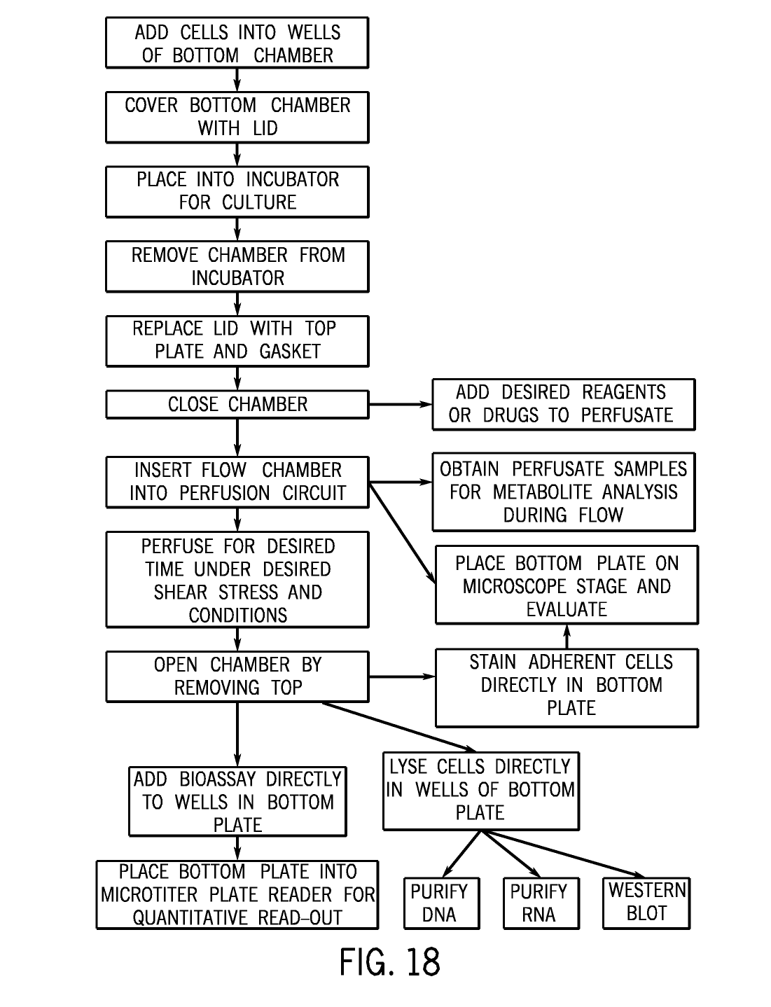

The flow chamber assembly may be used for culture of cells prior to a flow experiment. The sterile-packaged bottom plate of the flow chamber assembly is placed under a laminar flow hood and the package material removed. Adherent cells, e.g. endothelial progenitor cells, are pipetted into the rectangular wells of the bottom plate using sterile technique (FIG. 18). The wells are deep enough (1 cm) to allow for filling with a sufficiently large volume of cell culture medium for cell nourishment (well dimensions are 1 cm×2.7 cm×0.8 cm). If he bottom plate is further made out of transparent tissue-culture-treated polystyrene, which supports the adhesion, spreading and growth of adherent cells. After loading of the wells with cells and medium, the bottom plate is closed with a non-occlusive lid, that fits onto the bottom plate without preventing gas exchange (FIG. 18). The bottom plate with lid is placed into an incubator at 5% CO₂ and 37° C. The bottom plate and lid can be removed from the incubator at desired time intervals to observe the growth of cells in the transparent polystyrene wells using a light microscope.

### Operation of Flow Chamber Assembly for Flow Experiment

The layer of cells in the bottom wells are subjected to laminar flow fluid shear stress. The sterile-packaged top plate and gasket of the flow chamber are placed under a laminar flow hood and the package material removed. The gasket is placed onto the bottom plate in the depression between rim and bumpers using sterile gloves or forceps (FIG. 18). In another embodiment, the gasket is already assembled on the bottom plate by the manufacturer. The cell culture medium is removed from the wells and the cell layer optionally washed twice with phosphate buffered saline solution. Following, a predetermined volume of perfusate is added to the wells to cover the cell layer and evacuate (displace) air from the feeding channels in the top plate upon assembly of the flow chamber assembly. The top plate is now pressed down onto the bottom plate such that the flow protrusions of the top plate fit into the wells in the bottom plate. In one embodiment, the top plate is endowed with overhanging latches, said latches having teeth on the side facing the plate, and interlocking with teeth on the outside edges of the bottom plate when the top plate is pressed onto the bottom plate (FIG. 13). This closure mechanism functions as a snap seal, which keeps the gasket fully compressed between top and bottom plates. The height of the flow path inside the wells is constant and predetermined by the height of the bottom plate rim and bumpers touching the top plate undersurface,

### Operation of Flow Circuit for Flow Experiment

The assembled flow chamber assembly is inserted into a flow circuit, which in one embodiment comprises of a pump, tubing, pulse dampeners, fluid reservoirs and optionally a heating plate. In one embodiment of the flow chamber shown in FIG. 2, 12 wells are arranged in the bottom plate of the flow chamber and 2 of said wells are connected in parallel via fluid feeding channels in the top plate (one fluid feeding channel directs fluid to and from 2 flow protrusions, respectively). Therefore 6 separate circuits exist in parallel in one experiment per flow chamber assembly. A 6 channel extension pump head is used in conjunction with one peristaltic pump to direct the perfusate from 6 reservoirs to 6 pulse dampeners and into the 6 fluid feeding channels to 6 pairs of 2 wells and back into the reservoirs. in another embodiment, a 6 channel syringe pump is utilized instead of the peristaltic pump (and pulse dampeners). The 6 separate circuits may be filled with 6 different drugs and the effect of said drugs on stem cells tested under physiological fluid shear stress.

The desired shear stress acting tangentially on the cell layer inside the wells is chosen by programming the pump at a predetermined flow rate Q (for a specific width w of the wells and height h of the flow path inside the wells and viscosity μ of the perfusate). The viscosity of the perfusate is optionally increased to a predetermined value by the addition of dextran, which reduces the flow rate necessary to achieve a predetermined shear stress (see equation (4)). The flow chamber may be maintained at 37° C. by placing it onto a heating plate, and the pH of the perfusate is held constant by using a perfusate that at least in part consists of 4-(2-hydroxyethyl)-1-piperazineethanesulfonic acid (HEPES) buffer. Alternatively, the flow chamber assembly may be placed inside an incubator at 37° C. and 5% CO₂.

Operation of Flow Chamber Assembly with Plate Reader

The bottom part of the flow chamber is suitably compatible with microtiter plate readers and enables measurement of physical, chemical, or biological events inside the wells. Bioassays may be performed inside the wells after the exposure of cells to physiological fluid shear stresses and to drugs or toxic agents for toxicity studies (FIG. 18). After a flow experiment, the flow chamber is opened by breaking the snap seal and lifting the top plate from the bottom plate. A cell viability colorimetric assay, for example an MTT assay, is pipetted directly into the wells (FIG. 18). The bottom plate is covered with a lid and placed into an incubator for a predetermined period of time (FIG. 18). The lid is then removed and the bottom plate is inserted into a plate reader. Since the wells in the bottom plate are arranged in such a configuration that the beam paths of a standard microtiter plate reader transverse the wells, an immediate read-out for each well is obtained. 12 wells in the bottom plate are each trans-illuminated by by 3 separate beams per well and the averaged values of absorbance are an indicator of cell viability in the specific wells (and flow circuits).

Operation of Flow Chamber Assembly for Read-out with Microscope

The fully assembled flow chamber assembly may be placed onto a microscope stage while it is being perfused in a flow circuit (FIG. 18). The lower side of the flow protrusions suitably has minimal thickness of transparent polystyrene that light of a light microscope can transverse on its path from light source through top and bottom plates to microscope objective.

The wells in the bottom plate are constructed out of transparent material of such thickness that a standard 20× or 40× microscope objective (without oil immersion) allows visualization of adherent cells inside that well. The inside surfaces of the wells can be visualized with bright field illumination, phase contrast microscopy or fluorescent microscopy.

A microscope may be outfitted with an automatic stage that, when combined with a suitable software program, may scan the individual wells in the bottom plate of the flow chamber assembly in a predetermined matter to obtain images either before, after, or at various time points during flow experiments. Following this operation, the flow chamber assembly allows for time-lapse microscopy and live-cell imaging during a flow experiment. For use with upright microscopes, the flow chamber assembly may simply be inverted.

### Operation of Flow Chamber Assembly for DNA or RNA Expression Studies

In contrast to currently available microfluidic chambers, large numbers of cells may be harvested after flow exposure for analytical techniques, such as reverse transcription polymerase chain reaction (RT-PCR). Cells may be exposed to predetermined shear stresses and/or drugs. Total RNA is isolated for gene expression analysis (FIG. 18), e.g. with the Aurum Total RNA Mini Kit, Bio-Rad. cDNA may then be synthesized using the iScript kit (Bio-Rad). Quantitative RT-PCR may be performed with the Bio-Rad MyIQ iCycler to evaluate expression of predetermined genes of interest.

### Operation of Flow Chamber Assembly for Protein Expression Studies

Cells may be exposed to predetermined flow conditions. After exposure to flow, cells may be lysed directly inside the wells of the bottom plate. Protease inhibitors are then added to prevent the digestion of proteins by cells own enzymes. A Western blot or other protein analysis assay can then be performed to detect proteins of interest in the samples (FIG. 8).

### Operation of Flow Chamber Assembly for Drug Development, Toxicity Studies

The multi-well flow chamber assembly described herein may also be used for drug development or drug toxicity studies by adding different drugs or toxins to the flow circuits and testing cells' responses to agents of interest. Bioassays may be used in conjunction with a microliter plate reader for instantaneous read-out.

### Operation of Flow Chamber Assembly for Biofilm Adhesion Studies

The flow chamber assembly may be utilized for the evaluation of biofilm production and/or bacterial growth under different drug conditions or fluid shear stresses. The individual circuits are filled with bacteria and test drugs and the formation of biofilms in the individual wells and circuits is evaluated with phase contrast microscopy.

### Operation of Flow Chamber Assembly for Platelet Adhesion Studies

The adhesion of platelets (PLTs) to endothelial cells or matrix proteins inside the wells may be quantified using time-lapse microscopy. The study variables include compounds that inhibit platelet function or compounds that activate endothelial cells. Furthermore, the effect of different shear stresses on PLT adhesion may be studied.

### Operation of Flow Chamber Assembly for Stem Cell Differentiation Studies

The wells of the bottom plate may be filled with matrigel and stem cells seeded into said matrigel-filled wells. The chamber is then closed and perfused with shear stresses mimicking capillary flow conditions, At predetermined time periods, the cells are imaged to study their morphological changes during flow. After a predetermined time period, the chamber is opened and a bioassay added to the wells. Following, the bottom plate is covered with a lid, the chamber incubated for a predetermined time period and then evaluated with a microtiter plate reader.

### Operation of Flow Chamber Assembly for Other Life Science Applications

In addition to the examples of methods of using the flow chamber assemblies described above many other methods of using the flow chamber assembly will be apparent to those skilled in the art. The flow chamber assembly may be used for antimicrobial compound screening, microbial adhesion studies, chemical screening studies, screening compounds for impurities, dose-response studies, drug dissolution profile generation, drug target development, drug discovery research, evaluation of pathological states, viral studies, viral infection studies, matrix invasion studies, transmigration assays, cell growth and proliferation studies, cell migration assays, basement membrane formation and degradation research, cell-cell interaction assays, cellular adhesion strength quantification, cell spreading studies, dynamic cell adhesion studies, cell rolling studies, cell-ligand interaction studies, protein-cell interaction assays, protein-protein interaction assays, signal transduction studies, cell-signal pathway analysis, ion-flux studies, ELISAs, apoptosis assays, angiogenesis research, vasculogenesis research, cancer research, wound healing assays, thrombosis research, further nucleofection studies, transfection studies, molecular profiling, immunoassays, lipophilicity assays, enzyme kinetic studies, kinetic turbidimetric analyses, NADH and NADPH quantification, membrane fluidity studies, solubility assays, as well as other specific applications in the fields of cardiovascular research, immunology research, and parasitology.

1. A flow chamber assembly for subjecting cells or biological reagents to laminar flow conditions to provide a predetermined level of shear stress to the cells or biological reagents, the flow chamber assembly comprising:
a bottom plate having at least one well with a bottom surface adapted to receive the cells or biological reagents,
a top plate having at least one flow protrusion positioned and shaped to fit into the well of the bottom plate,
a sealing element positioned between the top plate and the bottom plate when the top plate and the bottom plate are attached, and
a flow path comprising a fluid feeding channel, an inflow bay, a laminar flow section, an outflow bay and a fluid exit channel;
wherein the fluid feeding channel is connected to the inflow bay formed between a first side of the well and a first side of the flow protrusion when the top plate and the bottom plate are attached,
the inflow bay is connected to the laminar flow section formed by the lower surface of the flow protrusion and the bottom surface of the well when the top plate and the bottom plate are attached,
the laminar flow section is connected to the outflow bay formed between a second side wall of the well and a second side of the flow protrusion when the top plate and the bottom plate are attached, and
the outflow bay is connected to the fluid exit channel such that when a perfusate is added to the flow chamber assembly via the fluid feeding channel the perfusate will flow to the inflow bay, through the laminar flow section and continue flowing out the outflow bay, and through the fluid exit channel,
the laminar flow section is suitable for subjecting the cells to laminar flow conditions when the top plate is attached to the bottom plate and the flow chamber assembly is in operation.

2. The flow chamber assembly of claim 1, wherein the sealing element is a gasket positioned between the top plate and the bottom plate and wherein the bottom plate has a rim to accept the gasket or wherein the sealing element is formed as part of the to plate or the bottom plate.

3. (canceled)

4. The flow chamber assembly of claim 1, further comprising an inlet port and an outlet port, wherein the inlet port is connected to the fluid feeding channel and the outlet port is connected to the fluid exit channel.

5. The flow chamber assembly of claim 4, wherein the inlet port, fluid feeding channel, fluid exit channel and outlet port are in the top plate such that when the flow chamber assembly is in operation the perfusate in the flow path begins in the top plate flows into the bottom plate at the inflow bay, flows back into the top plate at the outflow bay and exits the flow chamber assembly from the outlet port in the top plate.

6. The flow chamber assembly of claim 1, wherein the bottom surface of the well and the lower surface of the flow protrusion are made of an optically clear material suitable for optically monitoring the cells on the bottom surface of the well.

7. (canceled)

8. (canceled)

9. The flow chamber assembly of claim 1, wherein the bottom plate includes at least two wells and the top plate includes at least two flow protrusions and wherein at least a portion of the fluid feeding channel and at least a portion of the fluid exit channel are shared by the flow path when the flow chamber assembly is in operation.

10. (canceled)

11. (canceled)

12. The flow chamber assembly of claim 9, wherein the wells are connected to the fluid feeding channel in parallel, the fluid feeding channel and fluid exit channel volume is increased in the portion of the fluid feeding channel and the fluid exit channel shared by the flow path for the wells to allow substantially the same flow to each well when the flow chamber assembly is in operation.

13. The flow chamber assembly of claim 9, wherein the wells are connected in series to the flow path.

14. The flow chamber assembly of claim 1, wherein the wells are positioned to be compatible for use with a microliter plate reader, automated systems or robotics.

15. (canceled)

16. The flow chamber assembly of claim 1, wherein the top plate comprises a latching means and the bottom plate comprises a latching means that allow the top plate and the bottom plate to be removably attached.

17. (canceled)

18. (canceled)

19. (canceled)

20. The flow chamber assembly of claim 1, wherein the flow protrusion has at least two feet, the at least two feet rest on the bottom surface of the well when the flow chamber assembly is in operation and the at least two feet define the sides of the laminar flow section of the flow path and the height of the feet determines the height of the laminar flow section of the flow path when the flow chamber assembly is in operation.

21. The flow chamber assembly of claim 1, further comprising a pumping mechanism in the fluid feeding channel or connected to the fluid feeding channel.

22. The flow chamber assembly of claim 21, wherein the pumping mechanism is a magnetically driven impeller acting as a centrifugal pump.

23. (canceled)

24. The flow chamber assembly of claim 1, wherein the flow of a perfusate can be adjusted to produce a predetermined shear stress in the laminar flow section of the flow path in the flow chamber assembly during operation.

25. (canceled)

26. The flow chamber assembly of claim 1, wherein the bottom plate further comprises a bumper extending above the bottom plate, when the flow chamber assembly is in operation the top plate rests on the bumper and ensures the proper spacing is maintained between the lower surface of the flow protrusion and the bottom surface of the well.

27. A top plate for a flow chamber assembly for subjecting cells or biological reagents to laminar flow conditions to provide a predetermined level of shear stress to the cells or biological reagents, the flow chamber assembly including a bottom plate having a plurality of wells disposed therein, the top plate and the bottom plate capable of being removably attached, the top plate comprising:
at least one flow protrusion positioned and shaped to fit into the well of the bottom plate, and
a flow path comprising a fluid feeding channel, an inflow funnel, an outflow funnel, and
a fluid exit channel;
wherein the fluid feeding channel is connected to the inflow funnel and
the outflow funnel is connected to the fluid exit channel, and
wherein when the top plate is removably attached to the bottom plate to form the flow chamber assembly the inflow funnel and the side of the well create the inflow bay formed between a first side of the well and a first side of the flow protrusion,
the inflow bay is connected to the laminar flow section formed by the lower surface of the flow protrusion and the bottom surface of the well, and
the laminar flow section is connected to the outflow bay formed between a second side wall of the well, a second side of the flow protrusion and the outflow funnel,
the lower surface of the protrusion is suitable for subjecting the cells to laminar flow conditions when the top plate is attached to the bottom plate and the flow chamber assembly is in operation.

28. (canceled)

29. (canceled)

30. The top plate of claim 27, further comprising an inlet port and an outlet port, wherein the inlet port is connected to the fluid feeding channel and the outlet port is connected to the fluid exit channel.

31. (canceled)

32. The top plate of claim 27, wherein the top plate includes at least two flow protrusions and wherein at least a portion of the fluid feeding channel and at least a portion of the fluid exit channel are shared by the flow path when the flow chamber assembly is in operation.

33. (canceled)

34. (canceled)

35. The top plate of claim 32, wherein the fluid feeding channels are connected to at least two inflow funnels in parallel and the fluid exit channels are connected to at least two outflow tunnels in parallel, and the volume of the fluid feeding channel and fluid exit channel in the shared portion is increased to allow substantially the same flow to the laminar flow section when the flow chamber assembly is in operation.

36. (canceled)

37. (canceled)

38. (canceled)

39. The top plate of claim 27, wherein the wells are positioned to be compatible for use with a microtiter plate reader or robotics.

40. (canceled)

41. (canceled)

42. (canceled)

43. (canceled)

44. (canceled)

45. The top plate of claim 27, further comprising a pumping mechanism in the fluid feeding channel or connected to the fluid feeding channel.

46. The top plate of claim 45, wherein the pumping mechanism is a magnetically driven impeller acting as a centrifugal pump.

47. The top plate of claim 27, wherein the top plate comprises a latching means that allows the top plate and the bottom plate to be removably attached.

48. The top plate of claim 27, wherein the flow of a perfusate can be adjusted to produce a predetermined shear stress in the laminar flow section of the flow path in the flow chamber assembly during operation.

49. A flow chamber assembly comprising the top plate of claim 27, a bottom plate having at least one well with a bottom surface adapted to receive the cells or biological reagents, and a sealing element positioned between the top plate and the bottom plate when the top plate and the bottom plate are removably attached.

50. A system for subjecting cells or biological reagents to laminar flow conditions to provide a predetermined level of shear stress to the cells or biological reagents, comprising the flow chamber assembly of claim 1,
wherein the flow chamber assembly is connected to an external pump capable of pumping a perfusate through the flow chamber assembly and a reservoir connected in series to the flow chamber assembly and the pump or wherein the flow chamber assembly further comprises a pumping mechanism capable of pumping a perfusate through the flow chamber assembly and a reservoir.

51. (canceled)

52. (canceled)

53. The system of claim 50, wherein, the connections are made using tubing and the tubing is equipped with a sampling port allowing collection of the perfusate in the tubing after passage of the perfusate through the flow chamber assembly.

54. (canceled)

55. A method of using the system of claim 50 for subjecting cells or biological reagents to laminar flow conditions to provide a predetermined level of shear stress to the cells or biological reagents, the method comprising:
adding perfusate including cells or biological reagents to be analyzed to the well of the bottom plate;
attaching the top plate to the bottom plate of the flow chamber assembly;
pumping perfusate from a reservoir through the fluid feeding channel into the inflow bay, through the laminar flow section and back out through the outflow bay and the fluid exit channel to achieve laminar flow in the laminar flow section of the flow path.

56. The method of claim 55, wherein the predetermined shear stress can be applied to the cells or biological reagents in the laminar flow section of the flow path by adjusting a flow rate of the perfusate pumped through the flow chamber assembly and wherein the shear stress can be calculated according to equation (1):
$$Q = \frac{τ \cdot w \cdot {h}^{1}}{6 \cdot μ}$$
wherein Q is the desired flow rate, τ is the target shear stress acting tangentially on the cells, w is the width of the laminar flow section, h is the height of the laminar flow section and μ is the viscosity of the media.

57. (canceled)

58. (canceled)

59. The method of claim 56, further comprising assaying at least one property of the cells, biological reagents and/or the perfusate before, during or after exposure to laminar flow conditions.

60. (canceled)

61. (canceled)

62. (canceled)

63. (canceled)

64. (canceled)
